(19) United States
(12) Patent Application Publication
(10) Pub. No.: US 2003/0149289 A1
(43) Pub. Date: Aug. 7, 2003

(54) Carbonate catalyzed alcoholysis of triglycerides

(76) Inventor: Galen J. Suppes, Lawrence, KS (US)

Correspondence Address: HOVEY WILLIAMS LLP, Suite 400, 2405 Grand Blvd., Kansas City, MO, 64108, US

(21) Appl. No.: 10/375,215

(22) Filed: Feb. 26, 2003

Related U.S. Application Data
(63) Continuation of application No. 09/798,197, filed on Mar. 2, 2001, now abandoned.
(60) Provisional application No. 60/265,601, filed on Feb. 2, 2001.
(60) Provisional application No. 60/260,201, filed on Jan. 9, 2001.
(60) Provisional application No. 60/197,613, filed on Apr. 18, 2000.
(60) Provisional application No. 60/187,102, filed on Mar. 6, 2000.

(51) Int. Cl. C07C 51/43

(52) U.S. Cl. 554/174; 208/15

(57) ABSTRACT

A process for the carbonate-catalyzed alcoholysis of fatty acid glycerides is disclosed, wherein an alcohol (e.g., a C1-C6 mono-, di- or trialcohol) is reacted with a fatty acid glyceride (e.g., a plant or animal derived triglyceride) at elevated temperatures and superatmospheric pressures to give high yields of the corresponding ester. The preferred catalysts are the alkali metal, alkaline earth metal or zinc carbonates, with calcium carbonate being especially preferred because of its ready availability and physical integrity under reaction conditions. The alcoholysis reaction may be carried out in a single reactor, or on a continuous basis using a plug flow reactor.

### RELATED APPLICATION

This application is a continuation of application Ser. No. 09/798,197 filed Mar. 2, 2001 and claims the benefit of provisional applications Serial No. 60/265,601 filed Feb. 2, 2001, Serial No. 60/260,201 filed Jan. 9, 2001, Serial No. 60/197,613 filed Apr. 19, 2000 and Serial No. 60/187,102 filed Mar. 6, 2000.

### BACKGROUND OF THE INVENTION

1. Field of the Invention

The present invention is concerned with carbonate-catalyzed processes for the alcoholysis of glycerides to produce biodiesel or other useful products. More particularly, the invention pertains to such methods wherein an alkali metal, alkaline earth metal or zinc carbonate is used to catalyze the reaction between a glyceride and an alcohol at elevated temperatures (e.g., from about 160-300 C) and superatmospheric pressures to give very high alcoholysis yields.

2. Description of the Prior Art

Ester formation constitutes one of the most important classes of reactions in value-added processing of animal fats and vegetable oils. Typical schemes for ester formation include reactions of 1) an alcohol with an acid (esterification), 2) an ester with an alcohol (alcoholysis), 3) two different esters (transesterification), and 4) an ester with an acid (acidolysis). Of these reaction schemes, esterification and alcoholysis are by far the most important to the fat and oil industry since these make use of the fatty acid components that comprise most of fats and oils.

Conversion of fats and oils into monoglyceride and glycolate products has often been performed through hydrolysis followed by esterification. Although this multi-step scheme can be accomplished in one pot, the process 1) produces an aqueous waste stream containing both the hydrolysis catalyst and oil's/fat's natural glycerin, 2) requires drying of the alcohol-acid mixture to drive equilibrium toward ester formation, and 3) requires the removal of the homogeneous catalyst from the product.

Alcoholysis schemes have the advantage of requiring only one reaction step. Simple Bronsted bases like NaOH and KOH are widely used for alcoholysis of agricultural triglycerides with simple alcohols like methanol. This is the most common method for converting a triglyceride to a monoester (Swern, D. Ed, Bailey's Industrial Oil and Fat Products Volume 2, Fourth Edition, John Wiley & Sons, New York, 1982; Ma, F., et al., The Effects of Catalyst, Free Fatty Acids, and Water on alcoholysis of Beef Tallow, Trans. Am. Soc. Agric. Eng. 41:1261 -1264 (1998); Kawahara, Y., et al., Process for Producing Lower Alcohol Esters of Fatty Acids, U.S. Pat. No. 4,164,506 (1979); and Stoldt, S. H., et al., Esters Derived From Vegetable Oils Used as Additives for Fuels. U.S. Pat. No. 5,730,029 (1998)). While this process works well for mono-alcohols such as methanol and ethanol, we have found that little or no conversion occurs when using glycols as the transesterifying alcohol.

The catalytic activity of strong Lewis acids such as titanium (IV) alkoxides Ti(OR)₄ (Siling, M. I., et al., Titanium Compounds as Catalysts for Esterification and alcoholysis, Russ. Chem. Rev. 65(3):279-286 (1996); and Mascaretti, O. A., et al., Esterifications, alcoholysis, and Deesterifications Mediated by Organotin Oxides, Hydroxides, and Alkoxides, Aldrichimica Acta. 30(2):55-68 (1997)) can provide effective alternatives to traditional Bronsted base catalysts. However, many of these catalysts suffer from problems of cost, toxicity, and difficulty of removal from the product. Specifically, the active early transition metal catalysts tend to be poisoned by water, while the heavy metal catalysts are more durable, but quite toxic (Johnson, R. W., et al., Fatty Acids in Industry, Mercel Dekker, Inc, New York, 1989).

For many industrial applications heterogeneous catalysts (two phase catalysts) offer several advantages over homogenous catalysts (one phase catalysts), the largest being ease of separation from products. This alone provides sufficient motivation to investigate solid phase materials capable of catalyzing these reactions.

Calcium oxide is one of the few heterogeneous alcoholysis catalysts that is in common use (Johnson, R. W., et al., Fatty Acids in Industry, Mercel Dekker, Inc, New York, 1989) for monoglyceride production at temperatures of 200 to 220° C. with reaction times of 1 to 4 h. While the calcium oxide is largely a solid catalyst, the reaction mixture forms a slurry that requires a solid-liquid separation process; even agglomerate of CaO will easily breakup into fine particulates which are not easily separated from the liquid. Phosphoric acid is often used to neutralize residual basicity.

Alkaline earth metals are effective catalysts for certain ester formation or interchange reactions. Calcium and barium carbonates, acetates, oxides, and hydroxides are active catalysts for the production of sorbitol esters from free fatty acids (Swern, D. Ed, Bailey's industrial Oil and Fat Products Volume 2, Fourth Edition, John Wiley & Sons, New York, 1982). Other catalysts have also been studied for these alcoholysis reactions. Zinc oxide supported on alumina promotes alcoholysis reactions of alcohols higher than methanol (Stern, R., et al., Process for the Production of Esters from Vegetable Oils or Animal Oils Alcohols, U.S. Pat. No. 5,908,946 (1999)). U.S. Pat. No. 5,525,126 describes the use of the acetates of calcium and barium to the application of the alcoholysis of triglycerides with methanol at 200-250° C. Certain carbonate systems are also known to promote glycerolysis (Swern, D. Ed, Bailey's Industrial Oil and Fat Products Volume 2, Fourth Edition, John Wiley & Sons, New York, 1982; Stoldt, S. H., et al., Esters Derived From Vegetable Oils Used as Additives for Fuels. U.S. Pat. No. 5,730,029 (1998)).

U.S. Pat. No. 5,399,731 discloses that the alcoholysis of various triglycerides can be performed using C1-C5 monoalcohols in the presence of basic catalysts at +5-40° C. The preferred catalysts are sodium or potassium hydroxide or sodium or potassium alcoholates of C1-C5 monovalent alcohols. Although the carbonates are also incidentally mentioned, U.S. Pat. No. 5,730,029 discloses the use of alkali metal alcoholysis catalysts only at relatively low temperatures of under 100° C.

### SUMMARY OF THE INVENTION

The present invention overcomes the problems outlined above and provides an improved process for the alcoholysis of fatty acid glycerides wherein an alcohol is reacted with fatty acid glyceride(s) in a reaction mixture to yield corresponding esters via esterification and/or alcoholysis. The processes of the invention are carried out in the presence of a catalyst containing an alkali metal, alkaline earth metal or zinc carbonate at temperatures of from about 160-300° C., more preferably from about 200-280° C., and most preferably from about 240-270° C.

The alcoholysis reactions of the invention are normally carried out at superatmospheric pressures, typically ranging from about the bubble point of the reaction mixture up to about 100 bar, more preferably from about 1.1-1.5 times the reaction mixture bubble point, and most preferably at about 1.5 times the bubble point.

Although any one of the alkali metal, alkaline earth metal or zinc carbonate catalysts (or mixtures thereof) can be used to good effect, calcium carbonate is particularly preferred. This carbonate has a solid structure which does not readily disintegrate when contacted with a liquid in packed-bed columns or reactors. In this respect, calcium carbonate is greatly superior to the calcium oxides used in the prior art. The carbonates are true catalysts in the processes of the invention, i.e., they are not consumed during the course of the alcoholysis reaction. Moreover, the preferred calcium carbonate can be reused almost indefinitely without plugging or the like. The carbonate catalyst should be in particulate form with an average particle size of greater than about 0.5 mm, more preferably from about 1-4 mm.

The fatty acid glycerides are generally selected from the group consisting of vegetable and animal fats and oils and mixtures thereof, and usually contain a substantial fraction of triglycerides and at least about 20% by weight fatty acid moieties. While such naturally occurring glycerides are particularly suitable because of cost and availability, glycerides isolated or produced by means of interesterification or synthetically, such as trioleine, mono- and tripalmitine, tristearol, glycerol monooleate, glycerol monodistearate and the like can be used; also, waste oil products such as spent deep frying oil may be employed. Particularly preferred fatty acid glycerides are selected from the group consisting of oils or fats derived from soybean, palm, coconut, sunflower, rapeseed, cottonseed, linseed, caster, peanut, olive, safflower, evening primrose, borage, carboseed, animal tallows and fats, and mixtures thereof.

The alcohol fraction of the reaction mixture is selected from the group consisting of the C1-C6 straight or branched chain alkyl or olefinic mono-, di- and trialcohols, and mixtures thereof. Especially useful alcohols are selected from the group consisting of methanol, ethanol, propanol, butanol, ethylene glycol, diethylene glycol, triethylene glycol, glycerin and mixtures thereof.

The stoichiometry of the reaction mixture should be adjusted so that the ratio of ester functional groups to alcohol functional groups therein is from about 0.2-40, more preferably from about 0.45-10, and most preferably from about 1-4. An excess of alcohol serves to drive the alcoholysis to completion.

The reactions of the invention are preferably carried out at selected time and temperature levels for a period of from about 10 minutes to 10 hours, and more usually from about 15 minutes to 3 hours. Where the time-temperature parameters are properly selected, virtually complete alcoholysis can usually be accomplished within thirty minutes.

Where the processes of the invention are used to produce biodiesel or hydrophobic additives intended for use with a fuel, it is often preferred that a hydrophobic solvent such as a petroleum fraction having a 95% boiling point of less than about 670° F. be used as a solvent in the reaction mixture. For example, diesel fuel is an effective solvent when added a level of from about 2-90% by weight of the reaction mixture, more preferably from about 10-40% by weight thereof. It has been found that the use of a petroleum fraction as defined above such as diesel fuel is an effective solvent for the desired alcoholysis reactions that form hydrophobic fuel additives or blend stocks, inasmuch as the hydrophobic nature of the petroleum fraction assists in promoting selectivity away from hydrophilic products (e.g., diglyceride and monoglyceride), resulting in the formation of a purer glycerin byproduct. Such a fraction increases the activity of the hydrophilic products and drives the reaction equilibrium towards hydrophobic products with the desired effect of reducing the concentration of the more hydrophilic components in the product. This promotes yields to desired products and enhances physical separation characteristics of the glycerin-rich phase from the petroleum fraction-rich phase. Finally, the presence of a petroleum fraction results in more effectively repelling and expelling of water from the hydrophobic phase and thereby promotes conversion of any free fatty acids in the reaction mixture to the corresponding esters.

In order to further assist water removal, conversion of glycerides, and reducing costs of removing excess alcohol reagent, the alcoholysis reactions are preferably conducted in two serially interconnected flow reactors. During steady state operation, fresh triglycerides and recycled alcohol enter the first reactor wherein reaction conditions promote substantial conversion of glycerides forming glycerin byproduct. The glycerin byproduct is removed by liquid-liquid separation upstream of the second reactor. Alcohol reagent and the fatty acid product derivatives of the first reactor output stream are fed to the second reactor where the alcohol reagent increases conversions to desired ester products. High conversions in the second reactor is aided by removal of glycerin prior to the second reactor. Preferably the two flow reactors are packed with heterogeneous catalyst such as a metal carbonate. Alternatively, a soluble catalyst may be introduced with the alcohol reagent and recirculated with the alcohol from the second reactor to the first reactor.

### BRIEF DESCRIPTION OF THE DRAWINGS

FIG. 1 is a schematic block diagram of a packed-bed reactor system useful for glyceride alcoholysis in accordance with the invention;

FIG. 2 is a block diagram similar to FIG. 1, but illustrating a product recycle to reduce the reaction induction period;

FIG. 3 is another block diagram similar to FIG. 2, depicting product recycle and flush separation of water from the reaction;

FIG. 4 is a block diagram similar to FIG. 3, but adding an additional recycle and use of a petroleum fraction such as diesel as a solvent;

FIG. 5 is a block diagram of a packed-bed reactor system using countercurrent reactors;

FIG. 6 is a block diagram of another countercurrent reactor system;

FIG. 7 is a block diagram of a reactor capable of producing multiple products;

FIG. 8 is a schematic representation of a column-type packed-bed reactor useful in the invention; and

FIG. 9 is a block diagram of a reactor system for producing a nitrated transesterified product useful as a cetane improver.

### DETAILED DESCRIPTION OF THE PREFERRED EMBODIMENTS

As indicated previously, the processes of the invention can be carried out in simple one-pot reactors, or in more complex systems such as plug-flow reactors. For example, and referring to FIG. 1, a simple plug flow reaction system 10 includes a heating exchanger 12, furnace 14 and packed-bed reactor 16. A glyceride-containing oil or fat starting product is delivered via line 18 for passage in serial order through the exchanger 12, furnace 14 (natural gas, steam or electrical resistive) and reactor 16. An alcohol input line 20 delivers alcohol for mixing with the oil or fat starting product, thereby generating a reaction mixture which is heated within furnace 14 and delivered to reactor 16, to form the esterified final product. The product output from reactor 16 passes through line 22 back through exchanger 12 for heat recovery. The carbonate catalyst is placed within the reactor 16, so as to promote the desired catalytic contact between the reaction mixture and the carbonate. The configuration of FIG. 1 is the preferred embodiment when glycerides are allowed or preferred in the product.

FIG. 2 depicts another simple reactor system 24 having many of the components of system 10, namely heat exchanger 12, furnace 14, reactor 16, product input lines 18 and 20 and product output line 22. However, in this instance, the alcohol input line 20 is joined with glyceride input line 18 upstream of exchanger 12. Furthermore, a recycle line 26 serves to recycle a portion of the reaction product back into the reactor feed to reduce or eliminate reaction induction periods. Recycling may be achieved directly or indirectly by splitting the output product stream so that from about 5-95% of the reaction product is reintroduced into the reactor feed stream. A pump (not shown) is usually required to facilitate such product recycle, and the recycle may be introduced before or after passage through furnace 14.

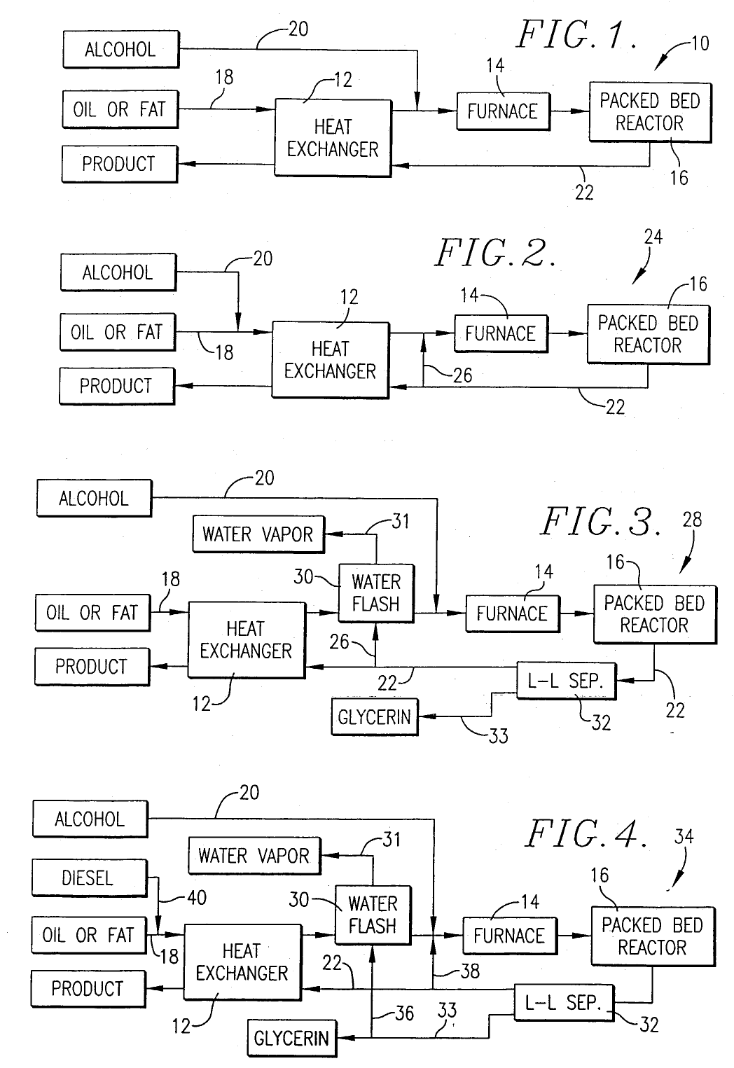

FIG. 3 illustrates a still further system 28 which again has many of the components of FIGS. 1-2, i.e., heat exchanger 12, furnace 14, reactor 16, glyceride input 18, alcohol input 20, product output line 22 and recycle 26. In this case, however, system 28 also includes a water flash unit 30 including an overhead flash output 31 interposed in glyceride input line 18 between exchanger 12 and furnace 14, with a liquid-liquid separator device 32 (having a glycerin output line 33) coupled within product output line 22. Note also that the recycle line 26 passes into the water flash unit 30. During esterification reactions, alcohols react with fatty acids derived from the glyceride input, forming water and the desired esters.

In this equilibrium-limited conversion water accumulation can limit conversion of the fatty acids to the corresponding esters. When water is present in the product stream at sufficient concentrations to limit reaction conversions (equilibrium limitation), the recycle stream is preferably flashed prior to recycle. Such flash separation is achieved by reducing the pressure below the bubble point of the product stream. Valving is the preferred method for reducing this pressure. As illustrated in FIG. 3, use of flash separation reduces the amount of water entering the reactor. Flashing of water is preferably performed prior to the addition of alcohol when the volatility of the alcohol is similar to or greater than the volatility of water, to reduce loss of alcohol in the flash overheads. Higher recycle rates result in lower water concentrations in the reactor effluent and higher fatty acid conversions. Known techniques may also be used to heat the alcohol input prior to the reaction and to recover alcohols exiting in the flash overheads from the unit 30. An advantage of the FIG. 3 embodiment is that a single flash separation serves to remove water from the incoming glyceride feed, as well as water formed during the esterification reaction.

FIG. 4 depicts a reaction system 34 having the exchanger 12, furnace 14, reactor 16, reactant inputs 18 and 20, product output line 22, water flash unit 30 and liquid-liquid separator 32. However, this embodiment adds a pair of recycle lines 36 and 38, as well as a diesel input line 40. In the ensuing discussion, reference is made to diesel as an additive in preferred processes; it is to be understood, however, that this is exemplary only, and that use can be made of a variety of petroleum products having a 95% boiling point of less than about 670° F. in such contexts. Line 36 extends from glycerin output line 33 to flash unit 30, whereas line 38 extends from product output line 22. In this embodiment, water is removed from the process selective to unreacted alcohol, this being accomplished by removing water from recycled glycerin. Additionally, the recycled glycerin provides a larger hydrophilic phase during the reaction that effectively removes water as it is formed, not unlike a desiccant. The liquid-liquid separator 32 separates the hydrophobic product phase from the hydrophilic phase that is rich in glycerin and water relative to the product phase. Introduction of diesel fuel through the line 40 enhances the separation and facilitates high distribution of water into the glycerin phase.

The embodiment of FIG. 5 illustrates a system 42 which may have many of the components of FIGS. 1-4 as desired, including glycerin and alcohol input lines 18 and 20, heat exchanger 12, furnace 14, diesel input line 40 and product output line 22. In this case, however, the system 42 includes a pair of packed-bed reactors 16a and 16b which are serially interconnected via line 44 and with an alcohol recycle line 32a extending from reactor 16b back to input 18 upstream of reactor 16a. Use of methanol and heterogeneous catalysts is preferred in this embodiment. Also, this scheme has a glycerin output 48 from the reactor 16a which passes through another heat exchanger 50; the fresh alcohol input line 20 also passes through the exchanger 50 and is coupled to line 44 intermediate the reactor 16a, 16b. In operation, an alcohol-rich phase with 0-90% of the ester product effluent of the reactor 16b (more preferably 5%-90%) is recycled and mixed with the glyceride feedstock in line 18 upstream of the first reactor 16a. Recycle stream flow is preferably controlled by conventional metering. In this embodiment, the feedstock is preferably soybean oil, rapeseed oil, beef tallow, lard, waste oils, mustard seed oil, palm oil, waste triglyceride oils, or a combination thereof, whereas the alcohol feed is preferably methanol or ethanol. Removal of the glycerin phase through line 48 is preferably level-controlled to allow removal of the physically separate glycerin phase without substantial removal of ester product with the glycerin; any conventional level controller known in the art can be used for this purpose including capacitance probes (not shown). Removal of the glycerin phase of reactor 16a prior to reactor 16b substantially reduces both the glycerin (bound and free) and water contents in reactor 16b; this increases overall conversions of free fatty acids and reduces the concentration of glycerides in the product. As before, diesel fuel may optionally be added via line 40 with the glyceride feedstock to induce better phase separation between the glycerin phase and the alcohol-rich phase in the second reactor 16b. The glycerin phase is the hydrophilic phase of the liquid-liquid equilibrium of the reactor effluents from reactor 16a; the glycerin phase is denser than the ester phase. As summarized in Example 7, lowered glycerin concentrations in reactor 16b reduces the tendency to form a second liquid phase high in alcohol concentration.

In order to facilitate formation of an alcohol rich phase from reactor 16b effluent, diesel may be used (from source 40a) as a solvent for the hydrophobic phase and lower temperatures are preferred. Temperatures of <75° C. are preferred in the product effluent of heat exchanger 12 to induce the desired phase behavior, with liquid-liquid separation in separator 32 and alcohol recycle via line 321. To induce the best liquid-liquid separation of methanol from the biodiesel, the US-2D:Biodiesel ratio entering the liquid-liquid separator should be greater than about 3:4. In addition, the ratio of methanol to US-2D should be greater than about 3:4 as well.

Alternatively, diesel can be added after the reactor at similar respective ratios the improvement being better liquid-liquid separation of alcohol from ester.

In order to reduce the amount of methanol exiting in the glycerin byproduct stream, the methanol recycle stream is preferably split so slightly greater than stoichiometric methanol (toward production of the desired ester product) is introduced into reactor 16a with the remaining methanol mixed with the methanol reagent feed. In this configuration, slightly greater than stoichiometric (relative to stream 18) methanol feed is preferred. The embodiment of FIG. 5 operated as described is the most preferred arrangement for producing biodiesel that is intended for immediate blending with diesel.

In an alternative embodiment, separation of alcohol from reactor 16b effluent in FIG. 5 is facilitated by mixing part of the glycerin effluent with the reactor 16b reactor effluent. This recycle of glycerin is preferably performed in a packed-bed reactor configuration where the absence of catalyst after reactor 16b does not provide a mechanism through which glycerin can drive the desired reaction backward forming glycerides. The advantages of this embodiment over the basic embodiment of FIG. 5 is that other factors driving liquid-liquid phase separation can be increased, including allowing separations at higher temperatures, use of ethanol as the alcoholysis reagent, and reducing or eliminating the need for diesel solvent. Such a method where the use of diesel as a solvent is eliminated is the most preferred means of producing diesel-free biodiesel.

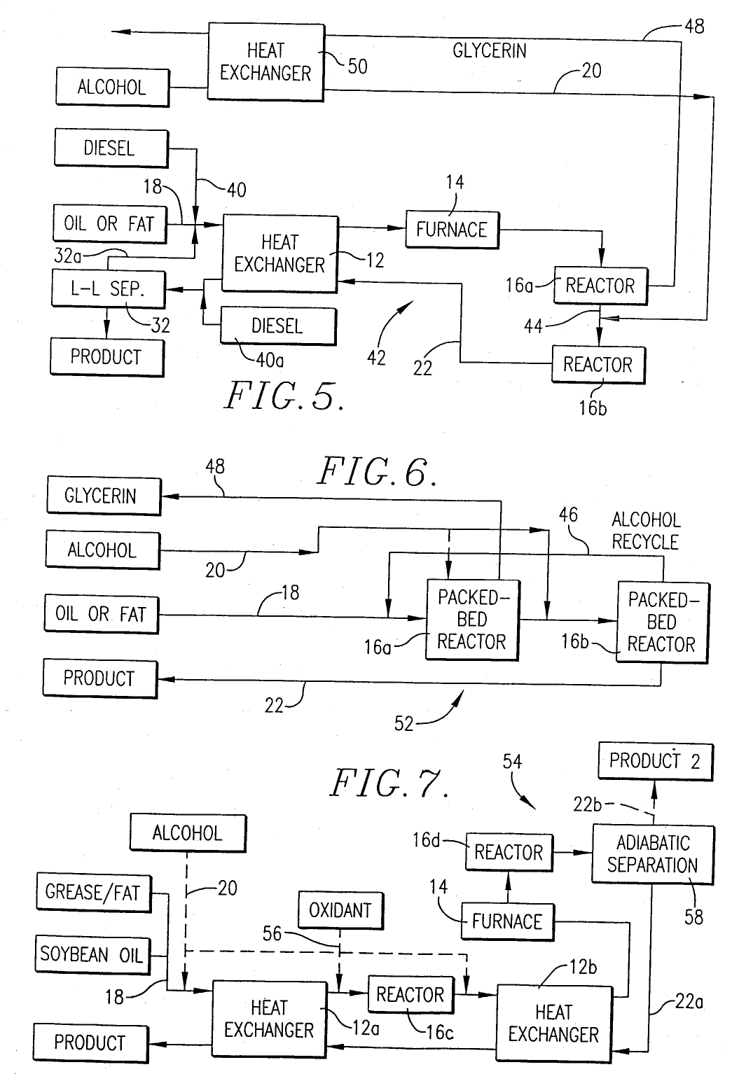

FIG. 6 illustrates a similar dual reactor system 52 employing the series-related reactors 16a and 16b in a broader scheme. This includes alternative recycle methods such as liquid-fluid separation possible at near-critical conditions. This figure also illustrates an alternative for addition of fresh alcohol, i.e., the alcohol may be added to the first reactor 16a as shown by the phantom line, or the alcohol may be added downstream of the first reactor.

An advantage of the dual-reactor systems of FIGS. 5 and 6 is that the exiting product through line 22 is exposed to a significant excess of alcohol while the main byproduct of the reactor series (the glycerin byproduct) is essentially free of the alcohol feedstock. Other reaction systems may have glycerin byproducts containing substantial amounts of feedstock alcohol that is preferably recycled—a costly operation. In the FIGS. 5-6 embodiment, the amount of alcohol feedstock delivered to the reactor 16b is near the stoichiometric amount (relative to glyceride input line 18) to convert all glycerides and free fatty acids to the corresponding ester. An additional advantage of the serial reactors is that water in the feed is substantially removed from the glycerin with the final reaction in the second reactor being free of the water removed from the glycerin of the first reactor. Of course, reduction of water promotes conversion of free fatty acids in the second reactor. At steady state, very little of the alcohol entering the second reactor 16b reacts; rather, most of it exits in the alcohol-rich phase from the second reactor and is recycled to react in the first reactor. A primary purpose of the second reactor 16b is to increase conversion, typically from <99% conversion in the first reactor 16a to >98% conversion in the second reactor 16b.

FIG. 7 depicts a system 54 designed to produce multiple products, using partial oxidation and alcoholysis to modify the triglyceride feedstock. It includes exchangers 12a and 12b, furnace 14, reactors 16c and 16d, glyceride input line 18, alcohol input line 20, optional oxidant input line 56, adiabatic separator 58 and first and second product output lines 22a and 22b. Dotted lines indicate optional configurations, depending upon the desired mode of operation. As shown, the triglyceride feedstock after passing through initial exchanger 12a may be oxidized using an oxidant such as ozone, oxygen or hydrogen peroxide, whereupon the feedstock passes through initial reactor 16c, secondary exchanger 12b, furnace 14, secondary reactor 16 and separator 58. Oxidation of the triglyceride feedstock selectively reacts with the carbon-carbon bonds of the triglycerides. The ester products are separated in separator 58 for individual recovery as shown.

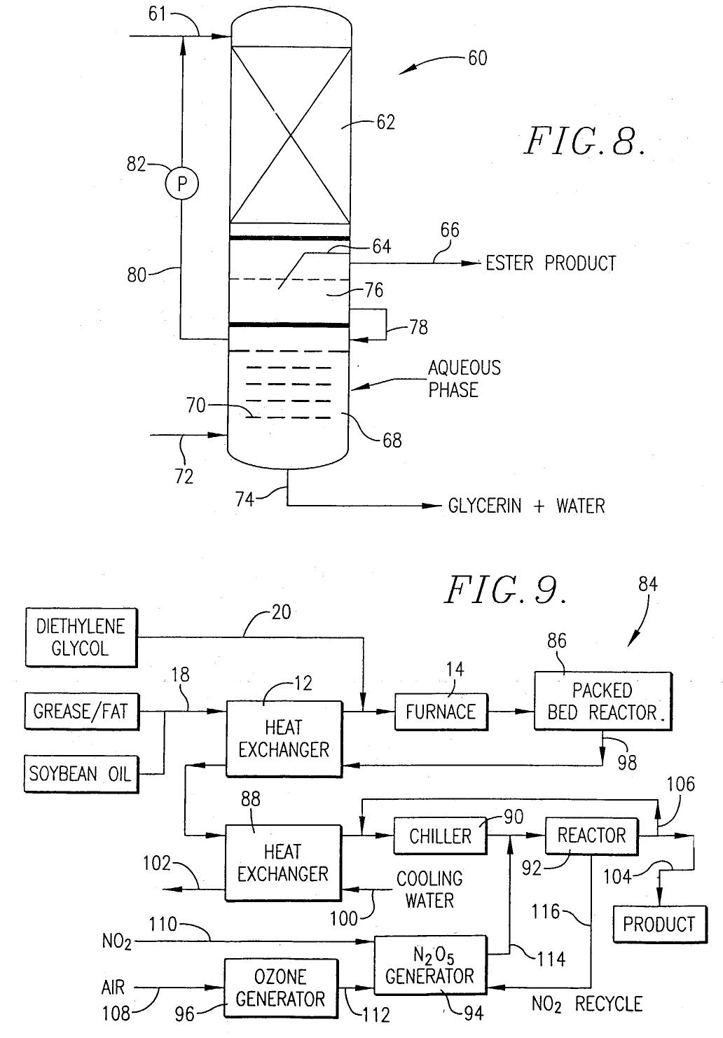

When lower alcohols such as methanol or ethanol react with triglycerides, excess alcohol reagent promotes conversion of the triglycerides. When using such excess alcohol, unreacted alcohol is preferably recycled. The column 60 depicted in FIG. 8 illustrates an embodiment in which a single column provides space for reaction, liquid-liquid separation, and vapor-liquid separation. The column 60 is an upright body having an uppermost input 61 for a reaction mixture of fat/oil and alcohol, with a packed bed reactor section 62 of carbonate catalyst (preferably calcium carbonate), a liquid-liquid separation baffle 64, an ester product output line 66 and a lower tray packing section 68. This packing 62 presents an upper input face and an opposed lower outlet face adjacent baffle 64. The section 68 has trays or packing 70 therein, a steam or heat input line 72 and a glycerin/water byproduct output line 74. Further, the overall column 60 has a coalesced packing section 76 above section 68, a liquid line 78 and a vaporized alcohol recycle line 80 equipped with pump 82. In the use of the column 60, a mixture of triglycerides and alcohol is mixed with recycle alcohol prior to entering the top of the column. This reaction mixture passes downwardly through the packed-bed reactor section 62 where conversion occurs. Fluids exiting the section 62 are physically separated into an upper ester product phase exiting through line 66 and a lower glycerin-rich phase. The latter proceeds through the section 68 where the pressure is reduced and/or heat is applied, thereby producing a vapor phase having a low glycerin concentration and a proportionately higher alcohol concentration. This vapor phase is recycled via line 80 as shown. Condensation and recycle of this vapor phase increases the alcohol concentration relative to the water concentration in the column reactor. The glycerin-rich liquid phase exits through line 74 whereupon water may be removed from the glycerin to allow recycle of substantially anhydrous glycerin. As will be appreciated by those skilled in the art, conventional control apparatus (not shown) is used with column 60 to control flow rates into, out of and between the different column sections.

Reactor 16a of FIGS. 5 and 6 is preferably of the type shown by FIG. 8 where the lower, packed section is not present with the glycerin effluent phase being taken at the bottom of the vessel and ester product being taken above the glycerin product and directly from below the baffle enhancing physical separation of the ester product from the glycerin. This embodiment is part of the most preferred schemes previously described for biodiesel production.

FIG. 9 illustrates a system 84 for the production of nitrated products useful as cetane or lubricity improvers. Broadly, the system 84 involves nitration of the alcoholysis products produced from the reaction of a glycol with a triglyceride, using dinitrogen pentoxide. Thus, the system 84 includes triglyceride and alcohol (glycol) input lines 18 and 20 together with heat exchanger 12 and furnace 14, all common to earlier embodiments. However, the system 84 includes a number of different components, secondary heat exchanger 88, chiller 90, reactor 92, N₂O₅ generator 94, and ozone generator 96. An output line 98 from reactor 86 passes through the exchangers 12 and 88 (the latter having a cold water inlet 100 and outlet 102), and thence through chiller 90 and reactor 92. A product output line 104 extends from the reactor 92, and a recycle line 106 is teed from line 104 back to line 98 upstream of chiller 90. An air input line 108 extends to ozone generator 96, while an NO₂ input 110 is directed to generator 94. Ozone and NO₂ enter generator 94 through inputs 110, 112, and N₂O₅ therefrom passes through line 14 for passage into reactor 92. NO₂ from the reactor 92 is recycled through line 116 back to generator 94. Reactor 86 is preferably packed with metal carbonate catalysts.

Promoters are commonly used to enhance the reactivity of known catalysts. Since calcium carbonate is sufficiently inexpensive and robust for use as a catalyst support, the reactivity of calcium carbonate can be readily enhanced by promoters. Promoting catalysts could be supported on calcium carbonate, supported jointly with calcium carbonate on a third substance/support, physically mixed with calcium carbonate, or combined with other methods known in the art. Useful promoters include metals, oxides, and other catalytic materials, especially those already known to promote the desired reactions including but not limited to silver, gold, platinum, iron, barium, palladium, bismuth, zinc, germanium, oxides of these metals, and mixtures of these metals.

In most chemical processes, the desired reaction and/or conversion requires several elementary reaction steps. For reactions were calcium carbonate promotes only part of the rate-determining processes, calcium carbonate can be used in combination with other catalysts. Other catalysts suitable for this application include metals, oxides, and other catalytic materials, especially those already known to promote the desired reactions including but not limited to silver, gold, platinum, iron, barium, palladium, bismuth, zinc, germanium, oxides of these metals, and mixtures of these metals.

In the case of reactions promoted by homogeneous acids, use of calcium carbonate catalysis after the acid-catalyzed reaction allows the calcium carbonate to serve two functions. The calcium carbonate can both neutralize residual acid and promote other reactions such as alcoholysis. For example, triglycerides containing free fatty acids can be esterified first with an acid catalyst and subsequently neutralized with a packed bed of calcium carbonate with alcoholysis being promoted by the calcium carbonate catalyst. Such a process comprises contacting a fluid containing residual acid and the glyceride-alcohol reactants with calcium carbonate after the acid catalyzed reaction at a sufficient combination of temperature and time to promote reaction.

Although acids can be added to promote esterification, calcium carbonate was shown to be effective in the presence of free fatty acids (see Example 4) unlike many alcoholysis catalysts that form soaps when contacted with free fatty acids.

Waste oils may provide a low-cost feed stock to the processes of the invention. Especially when these waste oils are separated from wastewater, they contain solids that do not easily separate from oils. The preferred method for separating the solids from the oils is to heat the mixture to a temperature >40° C. and preferably between 50° C. and 99° C. Advantageously, acid is added to this mixture to create a pH <5.0. Even more preferably, diesel is added to the mixture to act as a solvent that dissolves solids and reduces viscosity. After heating and mixing, the mixture solids are removed at the elevated temperatures by methods known in the art and the two liquid phases are physically separated—the acid and residual water form an aqueous phase. The clear organic phase is preferably processed using the process of the invention. The diesel may be added as part of the triglyceride feed stock preparation rather than as a reactor feed—it ultimately enters the reactor with the triglycerides and other fatty acid derivatives.

Of the metal carbonates, calcium carbonate provides a particularly good combination of low cost, catalytic activity, resistance to reaction with free fatty acids, and robust structure allowing use in a flow reactor. Metallic zinc has a comparable structure as calcium carbonate; however, it has an increased tendency to react with free fatty acids as illustrated in Example 9. To the advantage of zinc, it appears to be slightly more active toward catalyzing alcoholysis as illustrated in Example 8. Temperatures include 150-300° C. and preferably near 220°. In general, the conditions for using zinc as a catalyst are similar to the conditions for using calcium carbonate The strengths of calcium carbonate and zinc can be best realized by having a mixture of glyceride, free fatty acid, and alcohol first contact calcium carbonate to achieve conversions >90% and then contact zinc with a higher reactivity to force higher conversions. Similar approaches can be taken with other catalysts, like zinc, that have higher alcoholysis catalyzing capability than calcium carbonate, but also, exhibit a higher propensity for undesirable side reactions with free fatty acids.

In the discussion of the previous embodiments, recuperative heat exchangers are preferred when able to be used; however, other known methods to increase or decrease temperatures are within the ambit of this invention. The purpose of the furnaces in the previous embodiments is to increase the temperature of the reagents; other methods for increasing temperatures are also within the scope of this invention including but not limited heat exchange with utility-quality steam and use of electrical resistance heating.

The following examples set forth illustrative esterification/alcoholysis reactions in accordance with the invention. It is to be understood, however, that these examples are provided by way of illustration and nothing therein should be taken as a limitation upon the overall scope of the invention.

### EXAMPLE 1

In this series of tests, the glycerides in soybean oil and beef tallow were transesterified using various glycols and carbonate catalysts.

For the carbonate-based 250-mL batch-reactor studies, a 250-mL 3-neck round-bottom flask was charged with 30.0 g of oil or fat, a variable amount of glycol, and 2.0 g of catalyst. The contents were purged with oxygen-free, dry N₂ for 10 min prior to heating under a N₂ atmosphere. Stirring was maintained throughout the reaction by use of a magnetic stirrer. The temperature was allowed to increase to a maximum of 195-225° C. and held at around 210° C. for 2.5-3 h. After the contents of the flask cooled to near room temperature, the product oil was extracted into diethyl ether. The ether was washed four times with 1 N HCl (to remove the carbonate catalyst) and twice with distilled water. The ether layer was dried with anhydrous sodium sulfate and filtered. The solvent was removed by rotary evaporation. The reaction conversions were quantified by comparing integrations of ¹H NMR resonances characteristic of the starting oil/fat and the desired product ester.

Anhydrous ethylene glycol, diethylene glycol, and triethylene glycol were purchased from Aldrich (Milwaukee, Wis.). The glycols (reported purity of 99%) were used without further purification. Soybean oil, Food Club brand vegetable oil that is distributed by Topco Associates, Inc. (Skokie, Ill.), was purchased from a local grocery store. The reported composition for the soybean oil was 64% polyunsaturated fats, 21% monounsaturated fats, and 14% saturated fats. Beef tallow was supplied by Inland Products, Inc (Columbus, Ohio). Sodium carbonate, potassium carbonate, glycerin (certified ACS grade), and methanol (HPLC grade) were purchased from Fisher Scientific (Fair Lawn, N.J.). Zinc carbonate (Baker analyzed reagent grade) was purchased from J. T. Baker Chemical Co. (Phillipsburg, N.J.). Approximately 100 mesh calcium carbonate and calcium oxide used for batch reactor studies were obtained from Lancaster (Windham, N.H.).

As shown in Table 1, three hours at about 200° C. was sufficient to completely convert the triglyceride into the monoglycol product. Calcium, zinc, and magnesium carbonate all show good catalytic activity, producing a clean monoglycol product. Sodium and potassium carbonate, although active alcoholysis catalysts, were found to promote side reactions. The IR spectra of the products of these catalysts show the presence of a new carbonyl peak around 1710 cm⁻¹ and a broad hydroxyl peak characteristic of a carboxylic acid. This negative hydrolysis side reaction is a drawback to using these catalysts. The ¹H NMR spectra also show another interesting side reaction. The olefinic protons for fatty acid esters typically appear as a broad multiplet centered around 5.3 ppm. However, in the products obtained from the alkali carbonate reactions, additional peaks in the ¹H NMR spectra appear at 6.40, 6.15, and 5.75 downfield of the broad olefin multiplet. The position of these new olefin resonances suggests that the double bonds are migrating into conjugation with the carbonyl group.

The following Table 1 sets forth the results of these tests.

TABLE 1
Batch reactor studies on alcoholysis of soybean oil and beef tallow.

| Reactor | | | | Molar Ratio | Time | Temp. | Yield | Conversion |
|---|---|---|---|---|---|---|---|---|
| size (mL) | Fat/Oil | Alcohol^{a} | Catalyst | (Lipid:ROH:Cat.) | (h) | (° C.) | (g) | (%) |
| 250 | Soybean Oil | DEG | none | 1.72:14.2:1.0 | 2.5 | 205 | N/A | <5 |
| 250 | Beef Tallow | DEG | none | 1.75:14.2:1.0 | 2.5 | 205 | N/A | <5 |
| 50 | Soybean Oil | EG | NaOH | 0.92:9.16:1.0 | 24.0 | 150 | N/A | <5 |
| 50 | Soybean Oil | EG | H₂SO₄ | 10:20:1^{b} | 8.0 | 90 | N/A | <5 |
| 250 | Soybean Oil | EG | DOWEX 50W-X8 | 1.00:61.8:2.8^{c} | 48.0 | 120 | N/A | <5 |
| 50 | Soybean Oil | EG | (C₁₂H₂₇Sn)₂O | 62.0:1660:1.0 | 24.0 | 100 | — | >95 |
| 250 | Soybean Oil | GLY | CaO | 0.96:10.4:1.0 | 2.5 | 210 | — | >95 |
| 250 | Soybean Oil | GLY | CaCO₃ | 1.73:17.9:1.0 | 2.5 | 210 | 15 | >95 |
| 250 | Soybean Oil | DEG | CaCO₃ | 1.73:18.9:1.0 | 2.0 | 210 | 20.8 | >95 |
| 250 | Beef Tallow | DEG | CaCO₃ | 1.75:16.5:1.0 | 3.0 | 210 | 30.5 | >95 |
| 250 | Soybean Oil | DEG | Na₂CO₃ | 1.81:14.9:1.0 | 2.0 | 210 | — | >95 |
| 250 | Soybean Oil | DEG | K₂CO₃ | 2.37:19.5:1.0 | 2.0 | 210 | — | >95 |
| 250 | Soybean Oil | DEG | ZnCO₃ | 3.27:27.0:1.0 | 2.0 | 210 | 29.6 | >95 |
| 250 | Soybean Oil | DEG | MgCO₃ | 1.01:11.8:1.0 | 2.0 | 210 | — | >95 |
| 250 | Beef Tallow | TriEG | ZnCO₃ | 3.33:27.0:1.0 | 2.5 | 210 | 34.9 | >95 |

^{a}DEG = diethylene glycol, EG = ethylene glycol, GLY = glycerin, TriEG = triethylene glycol
^{b}approximate reaction conditions
^{c}2.8 g of the resin was used

### EXAMPLE 2

In these tests, a plug flow reactor was employed to transesterify soybean oil using various alcohols. A 20 mL reactor was packed with 14-20 mesh calcium carbonate catalyst obtained from Iowa Limestone Company of Des Moines, Iowa and continuous flow reactions were run at 4 and 18 minute residence times at 200 C. Greater than 95% conversion was achieved for alcoholysis with ethanol at 240 C and 18 minutes residence time. Reaction pressures varied from 10 to 100 bar with liquid phases being maintained at all times. The following Table 2 summarizes reactions performed in the packed-bed reactor.

TABLE 2

| | | | Soybean Oil |
|---|---|---|---|
| | Temperature | Residence | Conversion |
| | (° C.) | Time (min) | (%) |
| Equal Masses | | | — |
| soybean oil:glycerin | 200 | 4 | 17 |
| soybean oil:glycerin | 240 | 4 | <5 |
| soybean oil:glycerin | 240 | 18 | 18 |
| soybean oil:ethanol | 200 | 18 | <1 |
| soybean oil:ethanol | 240 | 4 | <5 |
| soybean oil:ethanol | 240 | 18 | <5 |
| soybean oil:ethylene glycol | 200 | 4 | <1 |
| soybean oil:ethylene glycol | 240 | 4 | <1 |
| soybean oil:ethylene glycol | 200 | 18 | 12 |
| soybean oil:ethylene glycol | 240 | 18 | 60 |
| Equal Masses | | | |
| soybean oil:glycerin | 200 | 18 | >95 |
| soybean oil:glycerin | 240 | 18 | >95 |
| soybean oil:glycerin | 260 | 18 | >95 |
| soybean oil:diethylene glycol | 200 | 18 | >95 |
| soybean oil:diethylene glycol | 240 | 18 | >95 |
| soybean oil:diethylene glycol | 260 | 18 | >95 |
| Mole Ratio 1.8:1 | | | |
| soybean oil:diethylene glycol | 200 | 18 | 35 |
| soybean oil:diethylene glycol | 240 | 18 | >95 |
| soybean oil:diethylene glycol | 260 | 18 | >95 |

Equal Masses in Methyl Ester of Soybean Oil (1 g oil, 1 g ethanol,
2 g methyl ester)

| | | | |
|---|---|---|---|
| soybean oil:ethanol | 200 | 18 | <1 |
| soybean oil:ethanol | 240 | 18 | 78 |
| soybean oil:ethanol | 260 | 18 | >95 |
| Soybean Oil | | | |

| | |
|---|---|
| 300 | polymerization occurred, |
| | plugging the reactor |

The same carbonate packing was used for a multitude of alcoholysis reactions, and no deterioration in performance was observed over the course of about 200 hrs. of reactions.

In the last test set forth in Table 2, soybean oil was contacted with calcium carbonate at temperatures ranging from 300-320 C at 100 bar. After about 5 minutes operation at this temperature, the reactor plugged, revealing a block paste reaction product. This demonstrates that at high temperatures undesirable polymerization reactions can occur.

### EXAMPLE 3

In order to enhance the conversion when reacting soybean oil with ethanol, a reactor feed containing 1 part soybean oil, 1 part ethanol, and 2 parts biodiesel (methyl ester of soybean oil) was fed to the plug flow reactor employed in Example 2. At 240 C and a 18 minute residence time the triglyceride conversion was 78% (compared to <5% without the biodiesel in the feed). At 260 C and an 18 minute residence time the triglyceride conversion increased to >95%.

### EXAMPLE 4

The impact of free fatty acids, that may be present in unprocessed fats/oils, on calcium carbonate catalyst was evaluated by adding fatty acid to sobyean oil. Thus, soybean oil was spiked with about 5 wt % fatty acids (prepared by hydrolysis of the same soybean oil). This mixture was reacted with diethylene glycol (1.8 mol diethylene glycol:1 mol oil) under the same conditions as those used in the batch-reaction. Spectra (IR and ¹H NMR) of the products of these reactions showed complete conversion of the starting oil to the desired glycol product. No evidence of residual free fatty acids or the corresponding calcium salt were seen in any of the spectra obtained. The product mixture was also titrated against KOH, and significant reductions in the amount of free fatty acids were observed. Therefore, the presence of free fatty acids in an oil/fat (such as that found in crude oils) does not poison the catalytic activity of calcium carbonate. In fact, titration results indicate that the free fatty acids were esterified over the calcium carbonate catalysts with a final free fatty acid concentration of less than 10% of the initial concentration. The high conversions of the free fatty acids was in part attributed to water being driven off in situ at the temperature and atmospheric pressure of this reaction.

Additional reactions were conducted with free fatty acid (FFA) added to soybean oil (SBO) with alcoholysis (and esterification) by diethylene glycol (DEG). These data is summarized below for batch reactions conducted at 210° C. and one atmosphere of pressure.

TABLE 3

| | | | |
|---|---|---|---|
| | | | CaCO₃ |
| DEG | SBO | FFA | Catalyst |

| | | | |
|---|---|---|---|
| (grams) | (grams) | SBO rxn | FFA rxn* |

| | | | | | |
|---|---|---|---|---|---|
| 30 | 0 | 30 | 3 | yes | yes |
| 30 | 0 | 30 | 0 | no | yes |
| 30 | 21 | 9 | 3 | yes | yes |
| 30 | 30 | 0 | 3 | yes | — |
| 30 | 30 | 0 | 3 | yes | — |
| 30 | 21 | 9 | 3 | yes | yes |
| 30 | 21 | 9 | 0 | no | yes |
| 30 | 30 | 0 | 0 | no | — |

*All FFA conversions < 90%

This data illustrates that even at 100% FFA, esterification proceeds at 210° C. The reaction proceeds in the absence of calcium carbonate. When SBO is present, even high concentrations of FFA do not substantially inhibit the ability of calcium carbonate to catalyze the alcoholysis reaction. The catalyst is necessary for alcoholysis as indicated by the reactions with SBO present but no calcium carbonate present. For these reactions, the conversions of FFA were all <90%.

Packed-Bed Flow Reactor Studies were also conduced on high FFA soybean oil. At a mass ration of 2:1:1, EtOH:SBO:Oleic Acid reacted at 260° C. and a residence time of 167 minutes an ˜60% FFA conversion and >95% SBO Conversion was obtained.

### EXAMPLE 5

In this test, a plug flow reactor having a volume of 200 ml (450 ml without packing) was packed with calcium carbonate, and a reaction mixture containing biodiesel, ethyl alcohol, soybean oil and free oleic acid was passed through the reactor. The conditions were as follows:

Reactant Mixture (mass basis)

| | |
|---|---|
| 1/3 | biodiesel |
| 1/3 | EtOH |
| 1/6 | soybean oil |
| 1/6 | oleic acid |
| Reactor and System Conditions | |
| Reactor Operating Temperature | 260° C. |
| System Operating Pressure (average) | 90 bar |
| System Flowrate (average) | ca. 1.2 mL/min |
| Amount of crude sample collected | |
| ca. 350 mL | |
| Washing and drying of crude product | |
| Sample washed twice with water. No significant prob- | |
| lem with emulsion and/or separation of emulsions | |
| Sample was dried overnight to obtain final product | |
| Free fatty acid results | |
| Reactant free fatty acid composition | 25% |
| Product free fatty acid composition | 10% |
| Free fatty acid reduction (single pass through reactor) | 60% |
| Conversion of triglyceride | >95% |

### EXAMPLE 6

In this test, the reactor and reaction conditions of Example 5 was used to evaluate the impact of using diesel solvent. The following sets forth the initial and final conditions.

Reactant Mixture (mass basis)

| | |
|---|---|
| 1/3 | #2 diesel |
| 2/9 | biodiesel |
| 2/9 | soybean oil |
| 2/9 | EtOH - Ethanol phase separated out |
| | of the reactants immediately when |
| | mixed (top layer) |
| Amount of crude sample collected | ca. 300 mL |

Appearance of crude sample

A two phase layer exists with ca. <50 mL of a lighter colored phase at the bottom, which may be a glycerin phase. A sample of the both of the crude phases is retained for further analysis. Further, the reddish color from the initial diesel oil is no longer present.

Washing and drying of crude product

Sample was initially washed with water. After setting for a number of hours (i.e. overnight) the water phase remained cloudy in appearance (and still is to this point). (I have not noticed this behavior in any of the other samples I have analyzed to date—that is, the wash is normally clear). Wash retained for further analysis if necessary. Second washing resulted in a significant amount of emulsion that did not separate. This mixture was then washed with ether which significantly reduced the amount of emulsion. Acetone was then added to the separated mixture to dissolve the remaining emulsion prior to drying. Sample was dried overnight to obtain final product.

Sample appearance and consistency

Sample is clear and light yellowish brown and the viscosity appears to be much lower than the previous samples over the last few weeks (i.e. ⅓ soy, biodiesel, & EtOH mixture as well as that with the free fatty acids).

NMR results

Sample contains 74% ethyl esters and 26% methyl esters.

There is a “trace” of glycerides (soybean oil, mono, di or tri) in the final product by NMR analysis.

### EXAMPLE 7

In this test, fundamental phase behavior is determined by mixing known quantities of glycerides, methyl esters, and alcohols. The biodiesel of this example is the methyl ester of soybean oil. These studies were conducted at atmosperic pressure and the indicated number of phases refers to the number of liquid phases.

Equal volumes of each component of the following binary systems provided the indicated phase behavior.

TABLE 4

| Components | | T (° C.) | Phases |
|---|---|---|---|
| Methanol | Biodiesel | 25 | 1 |
| Methanol | Canola Oil | 25 | 2 |
| Methanol | US-2D | 25 | 2 |
| Ethanol | Biodiesel | 25 | 1 |
| Ethanol | Canola Oil | 25 | 2 |
| Ethanol | US-2D | 25 | 1 |

A repeat of the above phase behavior studies at 70° C. provided similar phase behavior. These results indicate that methanol is sufficiently more hydrophilic than ethanol and that methanol exhibits different phase behavior.

Equal volumes of each component of the following ternary systems provided the indicated phase behavior. US-2D is number 2 diesel fuel.

TABLE 5

| Components | | | T (° C.) | Phases |
|---|---|---|---|---|
| Methanol | Biodiesel | US-2D | 25 | 2 |
| Methanol | Canola Oil | US-2D | 25 | 2 |
| Methanol | Biodiesel | US-2D | 75 | 1 |
| Ethanol | Biodiesel | US-2D | 25 | 1 |
| Ethanol | Canola Oil | US-2D | 25 | 2 |
| Ethanol | Biodiesel | US-2D | 75 | 1 |

An observation in addition to the phase behavior data of the above table is that the mixture containing 2 mL each of methanol, biodiesel, and US-2D has an upper methanol phase that is about 2 mL and contains most of the methanol. When an additional 2 mL of biodiesel is added to the mixture, the upper methanol phase is reduced to about 0.7 mL indicating that the biodiesel is taking in a substantial portion of the methanol into the hydrophobic phase. When an additional 2 mL of US-2D is added, the upper methanol phase volume increases again to about 1 mL; less than the 2 mL for the equal volume mixture. These data indicates that to induce a methanol rich phase in the presence of biodiesel, the ratio of US-2D:Biodiesel should be greater than about 3:4. In addition, the ratio of methanol to US-2D should be greater than about 3:4 as well.

Unreacted ethanol was largely immiscible with the product of Example 6 containing diesel and biodiesel, this was due to the presence of glycerin. Glycerin induces a hydrophilic phase when contacting biodiesel; this hydrophilic phase extracts most of the ethanol from the biodiesel.

The following mixtures contain equal volumes of the four components.

TABLE 6

| Components | | | | T (° C.) | Phases |
|---|---|---|---|---|---|
| Methanol | Biodiesel | US-2D | Glycerin | 25 | 2 |
| Methanol | Canola Oil | US-2D | Glycerin | 25 | 2 |
| Methanol | Biodiesel | US-2D | Glycerin | 75 | 2 |
| Ethanol | Biodiesel | US-2D | Glycerin | 25 | 2 |
| Ethanol | Canola Oil | US-2D | Glycerin | 25 | 2 |
| Ethanol | Biodiesel | US-2D | Glycerin | 75 | 2 |

For the embodiment of FIGS. 8 and 9 incorporating addition of alcohol between reactors and recycle of the ethanol phase after the second reaction, the output stream from the second reactor would be low in glycerin. This phase behavior data indicates that both lower temperatures (<75 C) and addition of diesel may be advisable to induce the second liquid phase. This approach has advantages over the alternative approach of flashing ethanol from the second reactor effluent since flashing is considerably more energy and equipment intensive.

It is well known that electrolytes, including bases, acids, and soluble salts, promote formation of hydrophilic phases. Electrolytes will induce a hydrophilic phase similar to glycerin that will draw out much of the alcohol from the biodiesel phase. For mixtures containing both glycerin and electrolytes, less diesel is required to induce the hydrophilic phase than is required when glycerin alone is used.

### EXAMPLE 8

In this test, the impact of lower reaction temperatures is evaluated with potassium and zinc carbonates.

Reactions were conducted at 60° C. and ˜4 hours in 10 mL batch vials for the following systems:

TABLE 7

| | Reaction Mixture (approximate grams) | | | |
|---|---|---|---|---|
| | Soybean Oil | Methanol | Salt | Reaction) |
| CaCO₃ | 6 | 3 | 1 | no |
| ZnCO₃ | 6 | 3 | 1 | no |
| KOH | 6 | 3 | 1 | yes |
| K₂CO₃ | 6 | 3 | 1 | yes |
| Na₂CO₃ | 6 | 3 | 1 | no |
| none | 6 | 3 | 1 | no |

Reaction was determined qualitatively by observing phase behavior. Each mixture initially had an upper methanol layer of about 3 mL that had a low viscosity. Reaction was identified after the ˜4 hour residence time by the upper methanol phase going away and a lower, more-viscous glycerin phase appearing. Except for KOH, all salts were beyond their solubility limit—most of each of the salts were present as solids.

In addition to these reactions, a equal mass mixture of ethylene glycol and soybean oil was reacted in the presence of both ZnCO₃ and K₂CO₃ at 150° C. for 2 hours. HNMR confirmed reaction for this system.

The catalytic ability of these salts appears to be of two types: (1) homogeneous catalysis driven by soluble ions and (2) heterogeneous catalysis requiring higher temperatures. The homogeneous catalysis appears to be related to the solubility constant of the salts. The below table lists solubilities for the salts in grams per 100 grams of water (from the Handbook of Chemistry and Physics, CRC Press, 62nd edition, R. C. Weast and M. J. Astle).

TABLE 8

Solubility (g/100 g Water)

| | 25° C. | 60° C. |
|---|---|---|
| CaCO₃ | 0.0014 | 0.0018 |
| ZnCO₃ | 0.001 | |
| KOH | 107 | 178 |
| K₂CO₃ | 112 | 156 |
| Na₂CO₃ | 7.1 | 46 |
| MgCO₃ | 0.04 | 0.01 |

CaCO₃ and ZnCO₃ were evaluated at 150° C. to determine if these two heterogeneous catalysts were effective at this temperature. At the end of 2 hours reaction time, the methanol phase appeared largely unchanged as an upper layer having lower viscosity than the lower layer.

TABLE 9

| | Reaction Mixture | | | |
|---|---|---|---|---|
| | (approximate grams, 150° C.) | | | |
| | Soybean Oil | Methanol | Salt | Reaction Conversion |
| CaCO₃ | 6 | 3 | 2 | <5% |
| ZnCO₃ | 6 | 3 | 2 | <23% |
| | Reaction Mixture | | | |
| | (approximate grams, 180° C.) | | | |
| | Soybean Oil | Methanol | Salt | Reaction Conversion |
| Zn (20 mesh) | 6 | 6 | | 45% |
| ZnO | 6 | 6 | 1 | 45% |
| | Reaction Mixture | | | |
| | (approximate grams, 150° C.) | | | |
| | Soybean Oil | EG | Salt | Reaction Conversion |
| CaCO₃ + | 6 | 3 | 4 | 22% |
| ZnCO₃ | | | | |

### EXAMPLE 9

In this test, the tendency of metal carbonates and other potential catalysts to form soap with pure oleic acid was evaluated. Systems were mixed, held at 120° C. for about 2 hours, then cooled to ambient temperatures and evaluated.

TABLE 10

| | Reaction Mixture | | | | |
|---|---|---|---|---|---|
| | (approximate | | | | |
| | grams, 120° C.) | | | | |
| | Oleic Acid | Salt | Hot Observation | Ambient Observation | Soap Formation |
| CaCO₃ | 2 | 0.5 | Solids, Cloudy | Solids, Cloudy | low to none |
| ZnCO₃ | 2 | 0.5 | Clear | All Solids | very high |
| MgCO₃ | 2 | 0.5 | Solids, Cloudy | Solids, Cloudy | low |
| K₂CO₃ | 2 | 0.5 | Solids, Clear | All Solids | very high |
| CaO | 2 | 0.5 | Solids, Cloudy | Solids, Cloudy | low to none |
| Zn | 2 | 0.5 | Viscous, green | Solid Zn and Solid | acid changed |
| | | | tint liquid, solids | Acid Phase | uncertain of |
| | | | | | product |
| ZnO | 2 | 0.5 | Viscous Liquid, | Solid Acid Phase | acid changed |
| | | | Most ZnO | with Some ZnO | probably soap |
| | | | dissolved, Solids | Solids | |
| none | 2 | | Clear | Clear | none |

The samples that were cloudy at ambient observations had clear liquid phases after centrifuging. The soap formation was determined by changes in phase behavior as a result of mixing and heating as well as susceptibility of mixture to lather/foam when mixed with an equal volume of water and mixed.

This data indicate that not all metal carbonates resist saponification, but calcium carbonate and calcium oxide are quite resistant.

I claim:

1. In a process for the alcoholysis of fatty acid glycerides wherein an alcohol is reacted with a fatty acid glyceride in a reaction mixture to yield the corresponding ester, the improvement which comprises carrying out said reaction in the presence of a catalyst containing an alkali metal, alkaline earth metal or zinc carbonate and at a temperature of from about 160-300° C.

2. The process of claim 1, said temperature being from about 200-280° C.

3. The process of claim 1, said reaction being carried out at a pressure of from about the bubble point of the reaction mixture Up to about 100 bar.

4. The process of claim 3, said pressure being from about 1.1-1.5 times said bubble point.

5. The process of claim 1, said carbonate catalyst being calcium carbonate.

6. The process of claim 1, said fatty acid glyceride selected from the group consisting of vegetable and animal fats and oils and mixtures thereof.

7. The process of claim 6, said fatty acid glyceride selected from the group consisting of oils or fats derived from soybean, palm, coconut, sunflower, rapeseed, cottonseed, linseed, caster, peanut, olive, safflower, evening primrose, borage, carboseed, animal tallows and fats, and mixtures thereof.

8. The process of claim 1, including the step of carrying out said reaction for a period of from about 10 minutes to 10 hours.

9. The process of claim 1, the ratio of ester functional groups to alcohol functional groups in said reaction mixture being from about 0.2-40.

10. The process of claim 9, said ratio being from about 0.45-10.

11. The process of claim 1, said alcohol selected from the group consisting of the C1-C6 straight or branched chain alkyl or olefinic mono-, di- and trialcohols, and mixtures thereof.

12. The process of claim 11, said alcohol selected from the group consisting of methanol, ethanol, propanol, butanol, ethylene glycol, diethylene glycol, triethylene glycol, glycerin and mixtures thereof.

13. The process of claim 1, said carbonate being in particulate form with an average particle size of greater than about 0.5 mm.

14. The process of claim 13, said size being from about 1-4 mm.

15. The process of claim 1, said reaction being carried out in a packaged bed reactor, with said bed comprising said carbonate catalyst.

16. The process of claim 1, wherein said reaction mixture includes a petroleum fraction having a 95% boiling point of less than about 670° F.

17. The process of claim 16, said fraction comprising diesel fuel.

18. The process of claim 17, said diesel fuel being present at a level of from about 2-90% by weight of the reaction mixture.

19. The process of claim 18, said level being from about 10-40% by weight.

20. The process of claim 1, wherein said glyceride contains greater than about 20% by weight fatty acid moieties.

21. In a process for the alcoholysis of fatty acid glycerides wherein an alcohol is reacted with a glyceride to yield the corresponding ester and glycerin byproduct, the improvement which comprises the steps of carrying out said reaction in a first and second serially connected flow reactors generating respective first and second reactor output streams and wherein, during steady state operations, fresh glycerides are added to said first reactor, fresh alcohol is added to the process, at least a portion of said glycerin byproduct is removed upstream of said second reactor, and at least some of the unreacted alcohol from said second reactor is recirculated to said first reactor.

22. The process of claim 21, wherein said fresh alcohol is added to said first reactor output stream.

23. The process of claim 21, wherein a portion of the unreacted alcohol from the second reactor is recirculated to said second reactor.

24. The process of claim 21, wherein said glycerin byproduct is removed from said first reactor.

25. The process of claim 21, said alcohol being methanol.

26. The process of claim 21, said first and second reactors including therein an alkali metal, alkaline earth metal or zinc carbonate catalyst, and said reaction being carried out at a temperature of from about 160-300° C.

27. The process of claim 21, wherein said glyceride contains greater than about 20% by weight fatty acid moieties and the said metal carbonate catalyst is calcium carbonate.

28. The process of claim 21, including the step of adding diesel to said second reactor output stream to generate two liquid phases therein, one of said liquid phases being alcohol-rich.

29. The process of claim 27, including the step of adjusting the temperature of said second reactor output stream to a level of from up to about 75° C.

30. In a process for the alcoholysis of fatty acid glycerides wherein an alcohol is reacted with a glyceride to yield the corresponding ester and glycerin byproduct, the improvement which comprises the steps of carrying out said reaction in a first and second serially connected flow reactors generating respective first and second reactor output streams and wherein, during steady state operations, fresh glycerides are added to said first reactor, fresh alcohol is added downstream of said first reactor, at least a portion of said glycerin byproduct is removed upstream of said second reactor, and unreacted alcohol from said second reactor is recirculated to said first reactor, said fresh alcohol comprising methanol, and further including the steps of adding diesel to said second reactor output stream and adjsuting the temperature of the second reactor ouput stream to a level of up to about 75° C.

31. An alcoholysis reactor for reacting fatty acid glycerides and aclohol starting ingredients to yield the corresponding esters, and glycerin byproduct, said reactor comprising:
a reactor body having at least one starting ingredient input, an ester product output and a glycerin byproduct output;
a packed bed of alcholysis reaction catalyst between said input and said outputs and presenting an inlet face and an outlet face;
a liquid-liquid separation baffle proximal to the outlet face of said bed and said ester product output, said baffle operable to direct an ester product-containing phase from said bed to said ester product output.

32. The reactor of claim 31, including a vapor liquid separation section located between said baffle and said glycerin byproduct output.

33. In a process for the alcoholysis of fatty acid glycerides wherein an alcohol is reacted with a fatty acid glyceride in a reaction mixture to yield the corresponding esters the improvement which comprises carrying out said reaction at a temperature of from about 0-300° C. where a petroleum fraction having a 95% boiling point of less than about 670° F. is added to the reaction mixture.

34. The process of claim 33, said fraction comprising diesel fuel.
